(19) United States
(12) Patent Application Publication
(10) Pub. No.: US 2026/0057522 A1
(43) Pub. Date: Feb. 26, 2026

(54) INFORMATION PROCESSING SYSTEM, INFORMATION PROCESSING METHOD, AND CELL MANUFACTURING METHOD

(71) Applicants: CANON KABUSHIKI KAISHA, Tokyo (JP); CANON MEDICAL SYSTEMS CORPORATION, Tochigi (JP)

(72) Inventors: YUKITERU MASUDA, Kanagawa (JP); MIE OKANO, Kanagawa (JP); SHUHEI TOBA, Kanagawa (JP); YOJI YAMAMOTO, Tokyo (JP)

(21) Appl. No.: 19/304,292

(22) Filed: Aug. 19, 2025

(30) Foreign Application Priority Data
Aug. 23, 2024 (JP) 2024-143443
Mar. 19, 2025 (JP) 2025-044614

(51) Int. Cl. G06T 7/00; C12M 1/00; C12M 1/34; C12M 1/36

(52) U.S. Cl. CPC G06T 7/0016; C12M 41/36; C12M 41/48; C12M 47/04; G06T 2207/10016; G06T 2207/10056; G06T 2207/20081; G06T 2207/30024

(57) ABSTRACT

An information processing system, comprises: an image data acquiring section that acquires image data including a colony of pluripotent stem cells; a first information acquiring section that acquires first information regarding morphological features of the colony by inputting the image data to a learned model; and a second information acquiring section that acquires second information regarding a chromosomal aberration of the colony based on the first information. The learned model is obtained by learning with a dataset including image data of a plurality of colonies of pluripotent stem cells having a chromosomal aberration and image data of a plurality of colonies of pluripotent stem cells not having a chromosomal aberration.

### BACKGROUND

### Field of the Technology

The disclosure of this specification relates to an information processing system, an information processing method, a program, and a cell manufacturing method.

### Description of the Related Art

In quality control in cell culture, chromosomal aberration testing of cells is being performed with increasing frequency. It is known that chromosomal aberrations in cells can potentially cause the canceration of cells, and to ensure the quality of cultured cells, chromosomal aberration testing is effective. However, the G-Band method and the FISH method (fluorescence in situ hybridization) used in conventional chromosomal aberration testing are destructive tests, and because they also require time until the test results are available, real-time testing in the cell culture process has been difficult. For this reason, a technology is sought that non-destructively and rapidly determines chromosomal aberrations in cells by analyzing images of the cells. Japanese Patent Laid-Open No. 2021-085849 proposes a method that analyzes an image of fluorescently-stained cells and determines whether a chromosomal aberration is present, and Japanese Patent Laid-Open No. 2024-510103 proposes a method that calculates pre-defined morphological feature values from a cell image, and detects cells including chromosomal aberrations by clustering the cells based on the calculated morphological feature values.

However, the detection method described in Japanese Patent Laid-Open No. 2021-085849 is a method that involves invasion of the cells and also requires effort and time for staining. Furthermore, with the method described in Japanese Patent Laid-Open No. 2024-510103, because it targets single cells, applying it to a cell colony requires isolating cells from the cell colony, and testing in the state of a cell colony cannot be performed.

### SUMMARY

According to one aspect of the present disclosure, an information processing system is provided, comprising:
an image data acquiring section that acquires first image data including a colony of pluripotent stem cells;
a first information acquiring section that acquires first information, which is information regarding morphological features of the colony of pluripotent stem cells included in the first image data, by inputting the first image data acquired by the image data acquiring section to a learned model obtained by learning with a dataset including image data of a first image group including a colony of pluripotent stem cells having a chromosomal aberration, and image data of a second image group including a colony of pluripotent stem cells not having a chromosomal aberration; and
a second information acquiring section that acquires, based on the first information, second information, which is information regarding a chromosomal aberration of the colony of pluripotent stem cells included in the first image data.

According to another aspect of the present disclosure, an information processing method is provided, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration; and
a second information acquiring step of acquiring, based on the first information, second information, which is information regarding a chromosomal aberration of the colony of pluripotent stem cells included in the first image data.

According to yet another aspect of the present disclosure, a cell manufacturing method is provided, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
a classifying step of classifying the colony of pluripotent stem cells included in the first image data, based on the second information.

According to yet another aspect of the present disclosure, a cell manufacturing method is provided, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
an eliminating step of eliminating a cell colony having a chromosomal aberration from the colony of pluripotent stem cells included in the first image data, based on the second information.

Features of the present disclosure will become apparent from the following description of embodiments with reference to the attached drawings. The following description of embodiments is described by way of example.

### BRIEF DESCRIPTION OF THE DRAWINGS

FIG. 1 is a diagram illustrating an example of a configuration of an information processing apparatus according to a first embodiment of the present disclosure.

FIG. 2 is a flowchart illustrating a sequence of processing performed by the information processing apparatus according to the first embodiment of the present disclosure.

FIG. 3 is a diagram illustrating an example of a configuration of an information processing apparatus according to a second embodiment of the present disclosure.

FIG. 4 is a flowchart illustrating a sequence of processing performed by the information processing apparatus according to the second embodiment of the present disclosure.

FIG. 5 is a flowchart illustrating a sequence of processing performed by an information processing apparatus according to a first modification of the second embodiment of the present disclosure.

FIG. 6 is a diagram illustrating an example of a configuration of an information processing apparatus and a cell culture apparatus according to a third embodiment of the present disclosure.

FIG. 7 is a flowchart illustrating a sequence of processing performed by the information processing apparatus and the cell culture apparatus according to the third embodiment of the present disclosure.

FIG. 8 is a diagram illustrating an example of a user interface that the information processing apparatus according to the third embodiment of the present disclosure notifies to a user.

FIG. 9 is a diagram illustrating an example of a user interface that the information processing apparatus according to the third embodiment of the present disclosure notifies to a user.

FIG. 10 is a diagram illustrating a confusion matrix of estimation results when performing chromosome 12 disomy/trisomy determination in a first example.

FIG. 11 is a diagram in which feature vectors of an intermediate layer are plotted in a two-dimensional space in the first example.

FIG. 12 is a diagram illustrating estimation accuracy for each number of days after seeding when performing per-pixel chromosome 12 disomy/trisomy determination in a second example.

FIG. 13 is a diagram illustrating a ground truth image and an estimation result when performing chromosome 12 disomy/trisomy determination on an image in which a colony group is independent in the second example.

FIG. 14 is a diagram illustrating a ground truth image and an estimation result when performing chromosome 12 disomy/trisomy determination on an image in which a fused colony is present in the second example.

### DESCRIPTION OF THE EMBODIMENTS

Hereinafter, some embodiments of an information processing apparatus, an information processing system, an information processing method, and a cell manufacturing method according to the present disclosure will be described with reference to the drawings. In the following embodiments, parts assigned the same reference numerals are assumed to perform similar operations, and redundant descriptions will be omitted as appropriate.

### First Embodiment

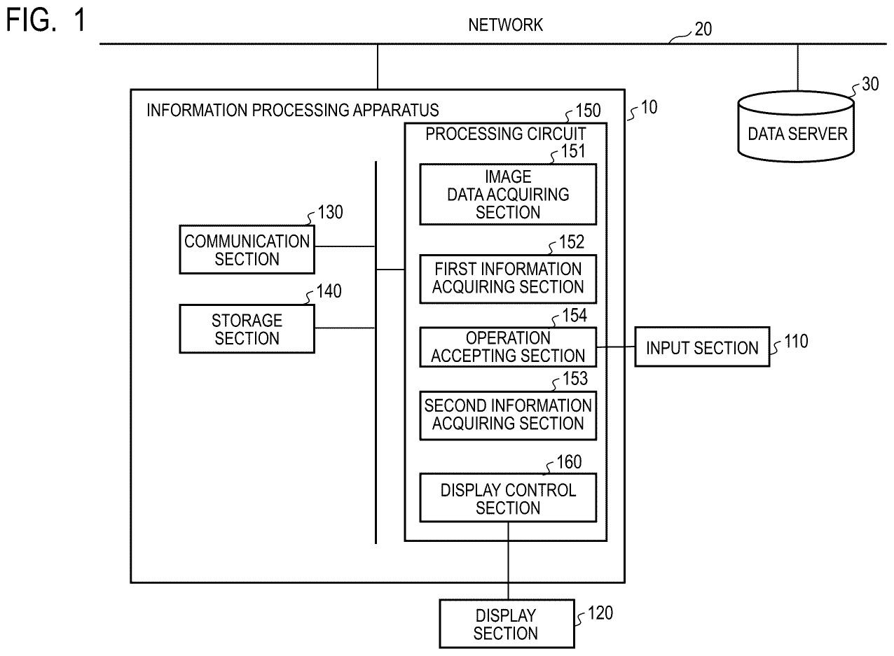

FIG. 1 is a schematic diagram illustrating an example of a configuration of an information processing system having an information processing apparatus according to a first embodiment. The information processing system illustrated in FIG. 1 has an information processing apparatus 10, an input section 110, and a display section 120. The information processing apparatus 10 and a data server 30 are configured to be connectable via a network. In FIG. 1, a configuration is illustrated in which the input section 110 and the display section 120 exist separately from the information processing apparatus 10, but the information processing apparatus 10 may have the input section 110 and the display section 120. As illustrated in FIG. 1, the information processing apparatus 10 according to the present embodiment includes a communication section 130, a storage section 140, and a processing circuit 150, and is connected to the data server 30 via a network 20. The configuration in FIG. 1 is an example, and the information processing apparatus 10 may be connected to other unillustrated apparatuses via the network 20 or an unillustrated communication cable, communication circuit, or the like. The data server 30, as illustrated in FIG. 1, may be provided in the information processing system separately from the information processing apparatus 10, or may be integrated with the information processing apparatus 10 as one of the elements constituting the information processing apparatus 10.

The input section 110 is an input section that a user operates when inputting various instructions and various types of information, and it transmits an electrical signal based on a user's input operation accepted by an operation accepting section 154 to the processing circuit 150 and the storage section (memory) 140. Specific examples of the configuration of the input section 110 include a mouse, a keyboard, a touch panel, a switch, a trackball, a microphone, a physical sensor, and the like. When the input section 110 is a touch panel, the input section 110 may also serve the function of the display section 120. The input section 110 does not require all of its input device components to be integrated with the information processing apparatus 10; for example, it may convert a remote input operation received via the network 20 into an electrical signal and transmit it to the processing circuit 150, the storage section 140, the display section 120, or the like.

A display control section 160 performs control to display various data on the display section 120, such as image data acquired by an image data acquiring section 151 in the processing circuit 150, information acquired by a first information acquiring section 152 and a second information acquiring section 153, and input operations input to the input section 110. Specific examples of the configuration of the display section 120 include a liquid crystal monitor, a touch panel screen having the functions of the input section 110 and the display section 120, and the like.

The communication section 130 communicates with other apparatuses through the network 20 and performs transmission and reception of information. Specific examples of the configuration of the communication section 130 include a network adapter, a network card, a NIC (Network Interface Controller), and the like.

The storage section 140 is a storage section that stores various data and various programs. Specifically, the storage section 140 is connected to, for example, the processing circuit 150, and stores image data acquired by the image data acquiring section 151, information acquired by the first information acquiring section 152 and the second information acquiring section 153, model data of a learned model, and the like. Specific examples of the configuration of the storage section 140 include semiconductor memory elements such as RAM (Random Access Memory) and flash memory, as well as hard disks, optical discs, and the like.

The processing circuit 150 has an image data acquiring section 151, a first information acquiring section 152, a second information acquiring section 153, an operation accepting section 154, and a display control section 160. In the present embodiment, each functional unit that is a component of the processing circuit 150 is recorded in the storage section 140 in a form that can be executed or read by a computer.

The image data acquiring section 151 acquires image data (first image data) of an image (first image) including a colony of pluripotent stem cells. In acquiring the image data, the image data acquiring section 151, for example, communicates with the data server 30 via the communication section 130 of the information processing apparatus 10 and the network 20, and acquires image data of an image including a colony of pluripotent stem cells stored in the data server 30. In the present embodiment, a colony of cells refers to a mass of aggregated cells formed after a single cell or a small cell cluster is seeded on a nutrient medium and the cells repeatedly proliferate and divide. The image including a colony of pluripotent stem cells is assumed to be captured using an imaging method such as phase contrast, differential interference, dark field, oblique illumination, or transmission defocus. The colony of pluripotent stem cells to be imaged in the present embodiment is preferably between the 5th and 9th day after seeding, or has a colony area of 50000 μm² or more.

The first information acquiring section 152 acquires first information, which is information regarding morphological features of the colony of pluripotent stem cells included in the image data acquired by the image data acquiring section 151. The first information can be acquired by the first information acquiring section 152 by being output after inputting the image data (first image data) including a colony of pluripotent stem cells acquired by the image data acquiring section 151 into a learned model described later. The first information is information regarding morphological features common to colonies of pluripotent stem cells included in a plurality of images of the same image group, out of a dataset including image data of a first image group containing a colony of pluripotent stem cells having a chromosomal aberration and image data of a second image group containing a colony of pluripotent stem cells not having a chromosomal aberration, which was used when training the model. The “morphological features common to colonies of pluripotent stem cells included in a plurality of images of the same image group” described here may be morphological features common to colonies of pluripotent stem cells included in a plurality of images of the first image group including a colony of pluripotent stem cells having a chromosomal aberration, or may be morphological features common to colonies of pluripotent stem cells included in a plurality of images of the second image group including a colony of pluripotent stem cells not having a chromosomal aberration. More specifically, the first information is, for example, specific colony shape distortions (such as circularity) or colony periphery shapes (such as the number of spikes existing on the periphery) or pixel biases common to colonies of pluripotent stem cells having a chromosomal aberration, or the average shape of colonies of pluripotent stem cells not having a chromosomal aberration, and these features are extracted by the learned model. The “plurality of” images described here may be a plurality of images that are a part of the image group, or may be all images of the same image group. Furthermore, the learned model used to acquire the first information is not limited to one that has been learned with the aforementioned dataset. For example, a model may be used that was learned using a dataset including image data of an image containing both a colony of pluripotent stem cells having a chromosomal aberration and a colony of pluripotent stem cells not having a chromosomal aberration, or image data of a colony containing both cells having a chromosomal aberration and cells not having a chromosomal aberration within the same colony. At this time, the learned model may extract, as the first information, features such as colony shape distortion or cell shape distortion common to regions having a chromosomal aberration among the colonies included in the input image.

As described above, the learned model that is executed by the processing circuit 150 of the information processing apparatus 10 and to which the image data acquired by the image data acquiring section 151 is input, is a model obtained by machine learning using as training data a dataset having image data of a first image group including a colony of pluripotent stem cells having a chromosomal aberration cell and image data of a second image group including a colony of pluripotent stem cells not having a chromosomal aberration, or a model obtained by machine learning using as training data a dataset having image data of an image group including both a colony of pluripotent stem cells having a chromosomal aberration and a colony of pluripotent stem cells not having a chromosomal aberration, and the like. At this time, for the ground truth labels of the dataset used for learning, labels such as “chromosomal aberration present,” “chromosomal aberration not present,” or a mixture of “present and not present” may be attached to each image according to the type of colony included in the image in the dataset, or regions with a chromosomal aberration and regions without a chromosomal aberration may be labeled for each pixel of the image. The first information, which is information regarding morphological features of the colony of pluripotent stem cells included in the first image data and is output from the learned model, is acquired by an unillustrated first information output section of the processing circuit 150 outputting it after the image data including a colony of pluripotent stem cells acquired by the image data acquiring section 151 is input to the learned model, and includes, for example, at least one of a feature vector resulting from a convolution operation on the first image data, a score for each pixel in the image including the cell colony, or a probability value for each pixel in the image including the cell colony. In such a case, the image data acquiring section 151 can also function as an image data input section.

The second information acquiring section 153 acquires, based on the first information acquired by the first information acquiring section 152, second information, which is information regarding a chromosomal aberration of the colony of pluripotent stem cells included in the image data including a colony of pluripotent stem cells that was input to the learned model. The second information is, for example, information regarding a determination of whether or not the colony of pluripotent stem cells has a chromosomal aberration (e.g., a determination result), a probability that the colony of pluripotent stem cells has a chromosomal aberration (a probability value), a clustering map with a colony of pluripotent stem cells estimated not to have a chromosomal aberration and a colony of pluripotent stem cells having a chromosomal aberration as separate classes, or information regarding a region estimated to have a chromosomal aberration in the colony of pluripotent stem cells (e.g., information indicating a region estimated to have a chromosomal aberration in the colony of pluripotent stem cells), and the like.

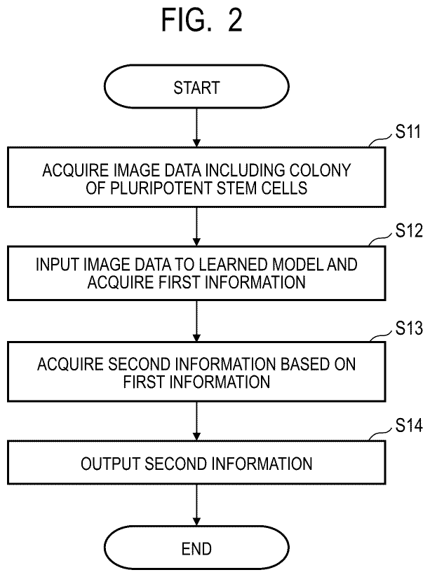

FIG. 2 is a flowchart illustrating a sequence of processing performed by the information processing apparatus 10 of the present embodiment.

### (S11)

In step S11, the information processing apparatus 10 receives, through the input section 110, information regarding image data to be acquired that includes a colony of pluripotent stem cells from a user, and transmits the information to the image data acquiring section 151. The image data acquiring section 151 may connect to the data server 30 through the communication section 130, and based on the information regarding the image to be acquired received from the user, acquire the image data to be acquired from the data server 30, or may acquire image data stored in the storage section 140, which will be described later. In acquiring the image data, the user may directly specify the image data to be acquired through the input section 110, or an image that matches conditions may be searched for from among an image group stored in the data server 30 based on conditions input by the user and acquired. The apparatus with which the image data acquiring section 151 communicates to acquire image data is not limited to the data server 30, and it may communicate with an unillustrated apparatus to acquire the image data. The acquisition of image data is not limited to being based on a user's instruction and may be performed by any other method; for example, when the image data of a captured image is saved to the data server, that image data may be acquired automatically. The image data acquiring section 151 transmits the acquired image data to the storage section 140, and the storage section 140 records the received image data including a colony of pluripotent stem cells. Although an example has been described here in which the image data acquiring section 151 acquires image data from the data server 30, the information processing apparatus 10 may have an imaging section, and the image data acquiring section 151 may acquire image data by capturing an image including a colony of pluripotent stem cells. An apparatus within the information processing system different from the information processing apparatus 10 may have an imaging section, and the image data acquiring section 151 may acquire image data captured by the imaging section of that apparatus.

### (S12)

The image data including a colony of pluripotent stem cells acquired in step S11 is input to the learned model, and the first information acquiring section 152 acquires the first information, which is related to morphological features of the cell colony, included in the input image data. Here, the information processing apparatus 10 does not need to perform the processing to generate the first information on the information processing apparatus 10 itself. For example, it may communicate with another arithmetic apparatus via the network 20, an image data acquiring section of the other arithmetic apparatus acquires image data including a colony of pluripotent stem cells, an operation for obtaining the first information (processing to generate the first information) is performed on the arithmetic apparatus, and the first information acquiring section 152 may acquire the first information by the information processing apparatus 10 receiving the first information generated as a result, via the network 20. In this way, even when the processing for generating the first information is performed by an arithmetic apparatus different from the information processing apparatus 10, the present disclosure's information processing system includes an information processing system having the arithmetic apparatus and the information processing apparatus.

The learned model can be trained, for example, using a known deep metric learning method that performs machine learning such that the feature distance between classes is increased, with image data including a colony of pluripotent stem cells having a chromosomal aberration and image data including a colony of pluripotent stem cells not having a chromosomal aberration as separate classes. More specific methods include those using Additive Angular Margin Loss or Triplet Loss as a loss function. In these methods, because a model is used that learns such that the feature distance between classes is increased, the learned model can extract the morphological features of the colony of pluripotent stem cells in each class as a feature vector in a high-dimensional feature space. That is, because the model learns to separate a colony of pluripotent stem cells having a chromosomal aberration and a colony of pluripotent stem cells not having a chromosomal aberration on the feature space, the extracted feature vector is considered to have information about morphological features specific to each class. Therefore, the first information acquiring section 152 can acquire the first information in the form of a feature vector regarding the morphology of the cell colony included in the input image data, by inputting image data including a colony of pluripotent stem cells to the trained learned model.

The learning method for the learned model exemplified above is one of the methods that can be used in the present embodiment, and the model may be trained using other learning methods, or the output of the learned model may be in a format other than a feature vector. The image data of the first image group including a cell colony having a chromosomal aberration and the image data of the second image group including a cell colony not having a chromosomal aberration used for training the learned model may include cell colonies obtained from multiple cell lines, not just a cell colony obtained from a single cell line. This makes it possible to separate specific morphological features caused by chromosomal aberrations from e.g. differences in cell colony morphology due to differences in cell lines, and an improvement in accuracy can be expected compared to when training with a single cell line.

### (S13)

In step S13, the second information acquiring section 153, based on the first information acquired in step S12, acquires second information, which is information regarding a chromosomal aberration of the colony of pluripotent stem cells, included in the image data including a colony of pluripotent stem cells that was input to the learned model in step S12. In the present embodiment, the second information acquiring section 153 acquires, as the second information regarding a chromosomal aberration of the colony of pluripotent stem cells included in the image data, for example, information regarding a determination of whether or not the colony has a chromosomal aberration (such as a determination result). As a specific method, the second information regarding the chromosomal aberration of the colony of pluripotent stem cells in the image data is acquired via processing by unsupervised clustering such as the k-means method, SVM (Support Vector Machine), a multilayer perceptron, a CNN (convolutional neural network), or the like, with the first information (feature vector) acquired in step S12 as input. At this time, the second information acquiring section 153 may use, in the SVM, multilayer perceptron, CNN, or the like used for acquiring the second information, one that has been learned using first information extracted from image data including different cell colonies of pluripotent stem cells acquired in advance, or may distinguish a colony of pluripotent stem cells with a chromosomal aberration from a colony of pluripotent stem cells without a chromosomal aberration using an unsupervised clustering method or the like.

In this way, by acquiring first information, which is information regarding morphological features of a colony of pluripotent stem cells from image data including a colony of pluripotent stem cells (for example, extracting as a feature vector the morphological features of at least one of, that is, one or both of, a colony of pluripotent stem cells having a chromosomal aberration and a colony of pluripotent stem cells not having a chromosomal aberration), and acquiring second information, which is information regarding a chromosomal aberration of the colony of pluripotent stem cells, with the first information (for example, the aforementioned feature vector) as input, it is possible to perform determination of a chromosomal aberration, etc., with an emphasis on the morphological differences of the colony of pluripotent stem cells caused by the chromosomal aberration, and an improvement in accuracy can be expected.

More specifically, a model learned by deep metric learning performs inference of a feature vector such that, with a colony of pluripotent stem cells having a chromosomal aberration and a colony of pluripotent stem cells not having a chromosomal aberration as separate classes, the same class becomes close on the feature space and different classes become far on the feature space. This makes it possible to learn a more robust representation for intra-class variation, even in a classification task with large individual variations like cultured cell colonies. In other words, even if data within the same class varies greatly, it becomes possible to represent them as the same class with similar feature vectors. By acquiring information regarding a chromosomal aberration of the colony of pluripotent stem cells (for example, information on whether or not the colony of pluripotent stem cells has a chromosomal aberration, such as a determination result) based on the feature vector obtained in this way, it becomes possible to construct a determination model with higher generalization performance compared to a machine learning model that directly determines from image data whether or not the cell colony has a chromosomal aberration (that is, a machine learning model that does not acquire first information).

By performing dimensionality reduction on the obtained feature vector by a method such as the t-SNE method (t-Distributed Stochastic Neighbor Embedding) and mapping it to a two-dimensional feature space, it is also possible to visually interpret how a colony of pluripotent stem cells having a chromosomal aberration and a colony of pluripotent stem cells not having a chromosomal aberration are separated and form clusters.

The processing to calculate a feature vector or the like as the first information, which is performed in step S12, and the processing to calculate a determination result or the like as the second information, which is performed in step S13, may be performed sequentially within a single machine learning model. As a specific example, multitask learning is performed with a single model, and both Additive Angular Margin Loss, which learns to increase the inter-class distance, and CrossEntropyLoss, which learns the class determination result, are calculated at different layers of the model, and the model is learned using the weighted sum of each as the final loss function. By executing the processing of step S12 and step S13 within a single model with such a multitask learning model, it is possible to output both, for example, a feature vector as the first information and a determination result as the second information end-to-end, with image data as input. It goes without saying that such a case is also included in the concept that the information processing apparatus has a first information acquiring section, and in such a case, the first information acquiring section can also be called a feature vector calculation section.

The second information acquired by the second information acquiring section 153 is not limited to information regarding a determination of whether or not the colony of pluripotent stem cells to be analyzed has a chromosomal aberration. For example, the second information acquiring section 153 may use, as the second information, information regarding a region of pluripotent stem cells having a chromosomal aberration in the image data including a colony of pluripotent stem cells acquired by the image data acquiring section 151 (for example, information indicating a region of pluripotent stem cells having a chromosomal aberration). Alternatively, the processing from step S11 to step S13 is repeated for an image group including colonies of pluripotent stem cells cultured in the same culture medium, that is, the first information is acquired by inputting an image group of colonies of pluripotent stem cells at different positions included in the same medium culture to the learned model, and the acquisition of the second information based on the acquired first information is repeated, and as a final result (information based on the second information), information regarding the proportion of colonies of pluripotent stem cells having a chromosomal aberration relative to the cell colonies existing in the same culture medium (for example, a statistical value of colonies of pluripotent stem cells having a chromosomal aberration relative to the colonies of pluripotent stem cells existing in the same culture medium, such as a proportion, a ratio, or a percentage) may be calculated. Information based on such second information is also acquired based on the acquired first information, and therefore is assumed to be included in the second information acquired based on the first information.

### (S14)

In step S14, the second information acquiring section 153 transmits the acquired second information to the storage section 140, and the storage section 140 performs recording of the received second information. The display control section 160 displays the second information acquired by the second information acquiring section 153 on the display section 120, such as a liquid crystal monitor or a touch panel screen. At this time, the display control section 160 may perform control to display information based on the first information (the first information or information generated from the first information) on the display section 120 together with the second information.

The processing of the information processing apparatus 10 is performed as described above. According to the present embodiment, features of a cell colony's morphology are extracted as a feature vector (first information), and by using those feature vectors as input, information regarding a chromosomal aberration of a colony of pluripotent stem cells (second information) is acquired. As a result, the learning model can focus more on the features of a cell colony whose morphology is caused by a chromosomal aberration, from among the variations in the morphological features of a colony of pluripotent stem cells, and it is expected that the accuracy of information regarding a chromosomal aberration, such as the result of a chromosomal aberration determination, will improve.

### Modification of First Embodiment—Utilizing Time-Series Data

The image data acquiring section 151 according to the first embodiment may acquire, for the image data including a colony of pluripotent stem cells to be acquired, the data as time-series image data (which may be called time-series image group data) that shows a series of morphological changes in the culture process of the cell colony by imaging the same colony of pluripotent stem cells time-serially at different imaging timings. Similarly, when acquiring the first information, a series of time-series image data may be input to the learned model as the image data including the colony of pluripotent stem cells to be analyzed. In this case, the learned model may output a group of feature vectors corresponding to each image as the first information for the series of time-series image data, and the second information acquiring section 153 may acquire the second information with that group of feature vectors as input. The learned model may output a single feature vector corresponding to the series of time-series image data, and the second information acquiring section 153 may acquire the second information with the output single feature vector as input. As a result, the information processing apparatus 10 according to the present modification can acquire the first information and the second information while also considering the time-serial morphological changes of the cell colony being analyzed, compared to the information processing apparatus 10 according to the first embodiment, and it is expected that the accuracy of the second information, such as a determination result, will improve.

### Second Embodiment—Utilizing Supplementary Information for Acquiring First Information

In the first embodiment, only the image data including the colony of pluripotent stem cells to be analyzed was input, and the first information acquiring section 152 acquired the first information regarding morphological features of the colony of pluripotent stem cells, and the second information acquiring section 153 acquired the second information, which is information regarding a chromosomal aberration of the pluripotent stem cells. On the other hand, in the second embodiment, supplementary information corresponding to the image data including each colony is additionally acquired, and with both this supplementary information and the image data including each colony as input, the first information acquiring section 152 acquires the first information regarding morphological features of the colony of pluripotent stem cells, and the second information acquiring section 153 acquires the second information, which is information regarding a chromosomal aberration of the cells.

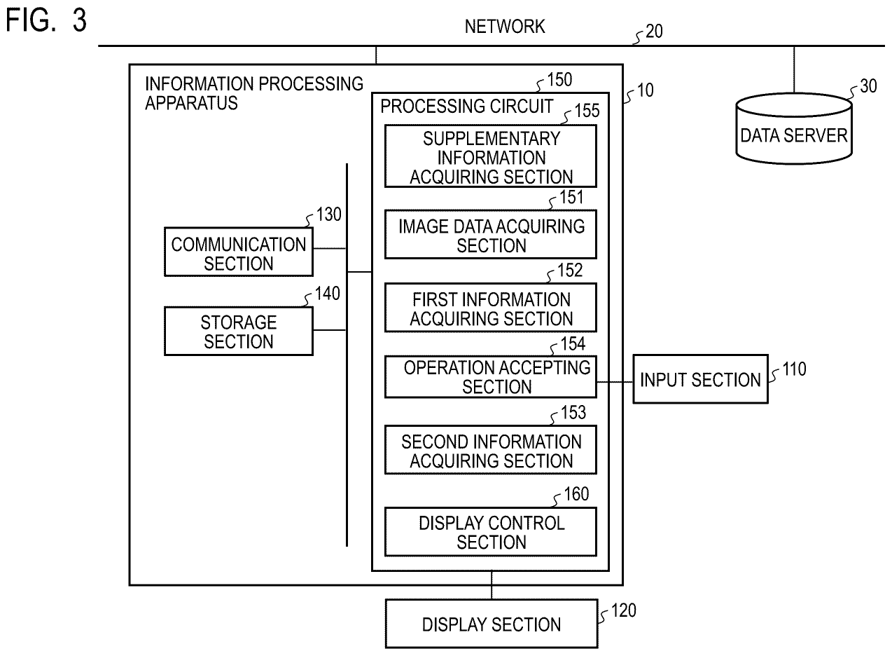

FIG. 3 is a diagram illustrating an example of a configuration of the information processing apparatus 10 according to the present embodiment. Parts of the present embodiment that are not particularly described are the same as in the first embodiment. As illustrated in FIG. 3, the processing circuit 150 according to the present embodiment has a supplementary information acquiring section 155, in addition to the components of the processing circuit 150 in the first embodiment. The supplementary information acquiring section 155 acquires supplementary information corresponding to the image data for the image data including each colony acquired by the image data acquiring section 151.

Specific examples of supplementary information include scalar values such as the amount of specific components in the medium during the culture of each colony of pluripotent stem cells (reactive oxygen species, lactic acid, etc.), pH, carbon dioxide concentration, and cell occupancy area ratio in the culture vessel. These can also be called scalar values of feature quantities related to the culture environment (which may be called scalar values related to the culture state). In addition, statistical values etc. based on an analysis of the image data including a colony of pluripotent stem cells (scalar values of statistical information), acquired by, for example, analyzing the texture feature pattern of the image including each colony of pluripotent stem cells with a Gray Level Co-occurrence Matrix (GLCM) or the like, may also be used as supplementary information. This supplementary information includes geometrical statistical values of the morphological features of the colony of pluripotent stem cells, and information indicating the degree to which the external environment is one in which chromosomal aberrations are likely to occur in the culture of the colony of pluripotent stem cells, and by analyzing this supplementary information in addition to the corresponding image data including the colony of pluripotent stem cells, highly accurate second information (such as a determination result) can be acquired. As a more specific example, the amount of metabolites such as reactive oxygen species and lactic acid in the medium components during culture is known as an indicator of whether the environment is one in which DNA damage due to environmental stress factors is likely to occur, and it can be expected that the determination accuracy will improve by inputting these as supplementary information.

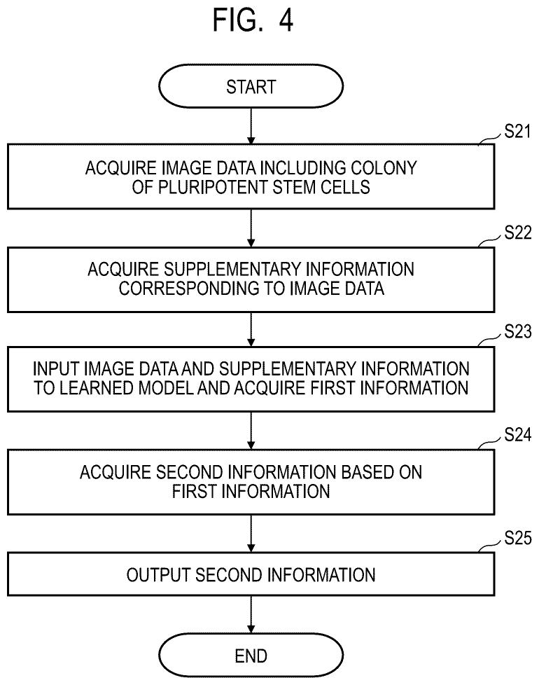

FIG. 4 is a flowchart illustrating the overall processing procedure performed by the information processing apparatus 10 according to the present embodiment. The processing of steps S21, S24, and S25 in this flowchart is identical to the processing of steps S11, S13, and S14 in FIG. 2, respectively, therefore description thereof is omitted. Hereinafter, using the flowchart of FIG. 4, only the differences from the first embodiment will be described.

### (S22)

In step S22, the supplementary information acquiring section 155 connects to the data server 30 through the communication section 130 and the network 20, and acquires supplementary information corresponding to the image data including a colony of pluripotent stem cells acquired by the image data acquiring section 151 in step S21. In acquiring the supplementary information, a type of supplementary information specified by the user via the input section 110 may be acquired, or the user may directly input the value of the supplementary information, and that value may be acquired as the supplementary information. The acquisition of supplementary information is not limited to being based on a user's instruction and may be performed by any other method; for example, when the image data of a captured image is saved to the data server, the supplementary information corresponding to that image data may be acquired automatically. The supplementary information acquiring section 155 associates the acquired supplementary information with the corresponding image data including a colony of pluripotent stem cells and transmits them to the storage section 140, and the storage section 140 records the received supplementary information in association with the corresponding image data including a colony of pluripotent stem cells. In the present embodiment, as a specific example of supplementary information, the medium pH value at the time of capturing the image data including a colony of pluripotent stem cells is assumed to be acquired.

### (S23)

Step S23 is a step corresponding to step S12 in the first embodiment. As a point of difference from step S12 of the first embodiment, the first information acquiring section 152 acquires the first information that was output by simultaneously inputting to the learned model, along with the image data including a colony of pluripotent stem cells acquired by the image data acquiring section 151, the medium pH value at the time the image including the cell colony was captured, which was acquired by the supplementary information acquiring section 155 in step S22, as supplementary information. The learned model is assumed to be a model that has been pre-trained under the condition that the medium pH value is also simultaneously input in addition to the image data.

In this way, by acquiring the feature vector, which is the first information, by inputting the medium pH value as supplementary information along with the image data including a cell colony, the reliability of the output second information is increased. It is expected that a colony of pluripotent stem cells whose medium pH value shows an abnormal value will have the effect of being more easily determined as a colony of cells with a chromosomal aberration on the feature vector. However, because there are also colonies whose medium pH value is abnormal but whose morphological features are distant from the morphological features of a colony of pluripotent stem cells having a chromosomal aberration, by acquiring the first information based on multiple types of information, such as information acquired from the image data of the colony of pluripotent stem cells and supplementary information such as the pH value, the reliability of the second information, such as the determination result of whether or not a chromosomal aberration is present, becomes higher than in the first embodiment.

### Modification of Second Embodiment—Using Supplementary Information Together With First Information

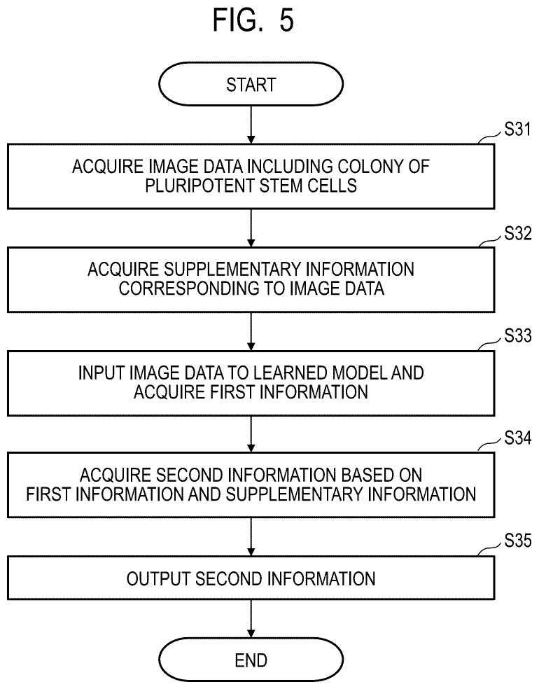

FIG. 5 is a flowchart illustrating a sequence of processing performed by an information processing apparatus 10 according to a modification of the second embodiment. The processing of steps S31, S32, and S35 in this flowchart is identical to the processing of steps S21, S22, and S25 in FIG. 4, respectively, therefore description thereof is omitted. Hereinafter, using the flowchart of FIG. 5, only the differences from the second embodiment will be described.

### (S33)

In step S33, when acquiring the first information, the first information acquiring section 152 inputs only the image data including a colony of pluripotent stem cells to the learned model and acquires the feature vector, which is the first information. In other words, the processing in step S33 is the same processing as step S12 of FIG. 2. At this time, the learned model is assumed to be a model that has been pre-trained to output the first information with only image data as input.

### (S34)

In step S34, the second information acquiring section 153, based on both the feature vector, which is the first information, and the pH value of the colony of pluripotent stem cells, which is the supplementary information acquired in step S32, acquires the determination result of whether or not the colony has a chromosomal aberration, which is the second information. At this time, the second information acquiring section 153 may acquire the second information by inputting both the feature vector, which is the first information, and the pH value of the colony into a single determiner, or may input the feature vector and the pH value of the colony into separate determiners, respectively, and integrate their output results to obtain the second information.

In addition to the setting of acquiring the second information based on the first information and the supplementary information described above, when integrating the output results of inputting the first information and the supplementary information to separate determiners, it is also possible to have a setting where if the scalar value of the supplementary information is outside a preset range, the result is determined as undeterminable, regardless of the result of inputting the first information to the determiner. In such a case, each setting can also be called a mode. More specifically, if the pH value of the colony is less than 6.9, the label “improper culture management” may be assigned as the second information. For example, it is known that for colonies of iPS cells, if the pH value of the medium becomes less than 6.9, gene damage increases significantly, and it is desirable from a quality control perspective to discard colonies of iPS cells cultured in that state, even if the morphological features related to chromosomal aberrations have not appeared. Therefore, by setting the second information for a cell colony whose pH value is outside a threshold to “improper culture management,” it can be determined or extracted as a cell colony that was not cultured in a proper culture process.

As described above, the information processing apparatus 10 of the present embodiment is an example that acquires the second information based on the first information obtained by inputting image data including a colony of pluripotent stem cells to a learned model, and supplementary information, instead of inputting the supplementary information to the learned model simultaneously with the image data. Furthermore, it is also possible to have a setting where if the scalar value of the supplementary information is outside a predetermined range, the second information is determined using only the supplementary information without using the first information.

### Third Embodiment—Combination with a Cell Culture Apparatus

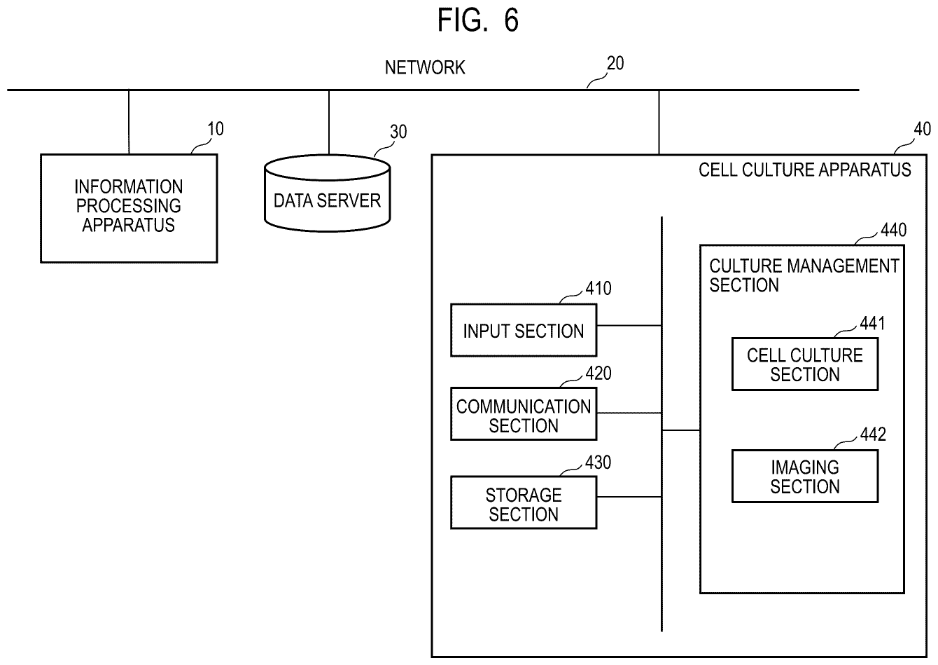

FIG. 6 is a diagram illustrating an example of a configuration of a cell culture system having an information processing apparatus and a cell culture apparatus according to a third embodiment. In the present embodiment, the information processing apparatus 10 described in the first embodiment and the second embodiment is connected to a cell culture apparatus 40 to monitor the occurrence of chromosomal aberrations in pluripotent stem cells. As illustrated in FIG. 6, the cell culture apparatus 40 includes an input section 410, a communication section 420, a storage section 430, and a culture management section 440, and is connected to the data server 30 and the information processing apparatus 10 through the network 20. The configuration in FIG. 6 is an example, and the information processing apparatus 10 or the data server 30 may be integrated with the cell culture apparatus 40. Among the components of the cell culture apparatus 40 illustrated in FIG. 6, the input section 410, the communication section 420, and the storage section 430 have the same functions as the components of the same name in the information processing apparatus 10 of the first embodiment, therefore description thereof is omitted.

The culture management section 440 has, for example, a cell culture section 441 and an imaging section 442. The cell culture section 441 has functions equivalent to those provided in a commercially available cell incubator, for example. Specifically, the cell culture section 441 has adjustment functions for temperature, CO₂ partial pressure, O₂ partial pressure, medium pH value, medium osmotic pressure, medium components, etc., to maintain the culture environment for culturing a cell sample, and a storage function for the cell sample. The cell culture section 441 may also periodically record the values of parameters indicating the culture environment and store them as data in the storage section 430.

The imaging section 442 has a function to image a cell sample being cultured in the cell culture section 441 and save it as image data in the storage section 430. Specifically, this function is realized by a phase-contrast microscope with an imaging function connected to the cell culture section 441, or the like. When capturing an image, the imaging section 442 may also associate metadata such as the imaging time and imaging position with the image data and save it in the storage section 430. Imaging by the imaging section 442 may be performed by a user manually operating a GUI or the like to specify the imaging timing, or may be performed automatically according to a pre-programmed schedule. When imaging by the imaging section 442, parameters of the culture conditions managed by the cell culture section 441 may be simultaneously acquired and saved in the storage section 430 in association with the image data.

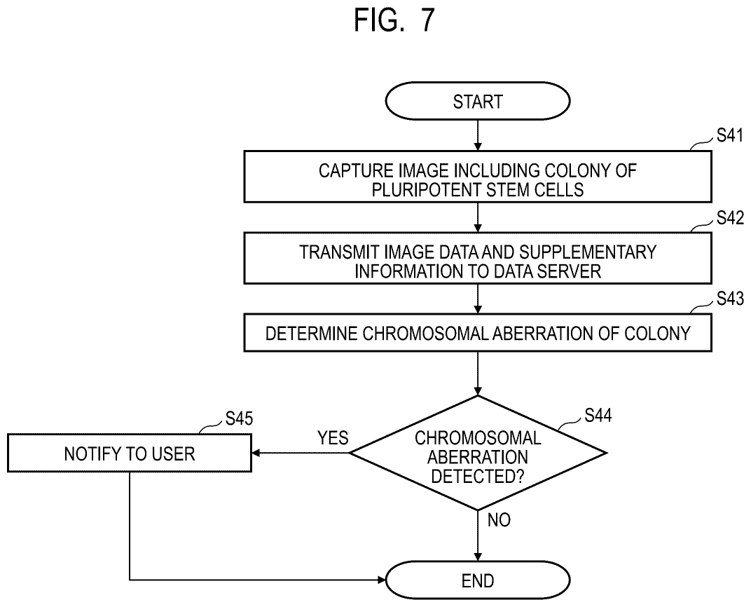

FIG. 7 is a flowchart of processing (monitoring of chromosomal aberrations during cell culture) performed by the information processing apparatus and the cell culture apparatus according to the present embodiment.

### (S41)

In step S41, the cell culture apparatus 40 captures an image including a colony of pluripotent stem cells being cultured in the cell culture section 441, using the imaging function of the imaging section 442. A case is exemplified where a commercially available 6-Well plate is used as the culture vessel and whole-well imaging is performed for each Well, but the culture method and imaging method in the present embodiment are not limited to this. The imaging section 442 automatically executes the imaging process according to a schedule input by the user in advance. Simultaneously with the imaging, metadata such as the Well number, imaging position, and imaging time of the captured image, and the values of parameters indicating the culture environment being monitored by the cell culture section 441 are simultaneously acquired, associated with the image data, and saved in the storage section 430.

### (S42)

In step S42, as soon as the imaging of the image including the colony of pluripotent stem cells is completed in step S41, the cell culture apparatus 40 transmits the image data including the cell colony and the data saved in association with it in the storage section 430 from the communication section 420 to the data server 30 via the network 20.

### (S43)

In step S43, the information processing apparatus 10 receives the image data and supplementary information data transmitted in step S42 from the data server 30 according to the means described in the first embodiment or the second embodiment, and acquires the second information, which is information regarding a chromosomal aberration of the colony of pluripotent stem cells. The specific means of information processing at this time has already been described in the first embodiment and the second embodiment, therefore description thereof is omitted. Using the colony's chromosomal aberration determination result as an example of the second information is also the same as in the first embodiment and the second embodiment.

### (S44)

In step S44, the information processing apparatus 10 acquires a determination result of whether there is a colony determined to have a chromosomal aberration as the second information, and if it is determined that no chromosomal aberration has occurred in the analyzed colony of pluripotent stem cells, the processing ends as is. If it is determined that a chromosomal aberration has occurred in the analyzed colony of pluripotent stem cells, the process proceeds to step S45.

### (S45)

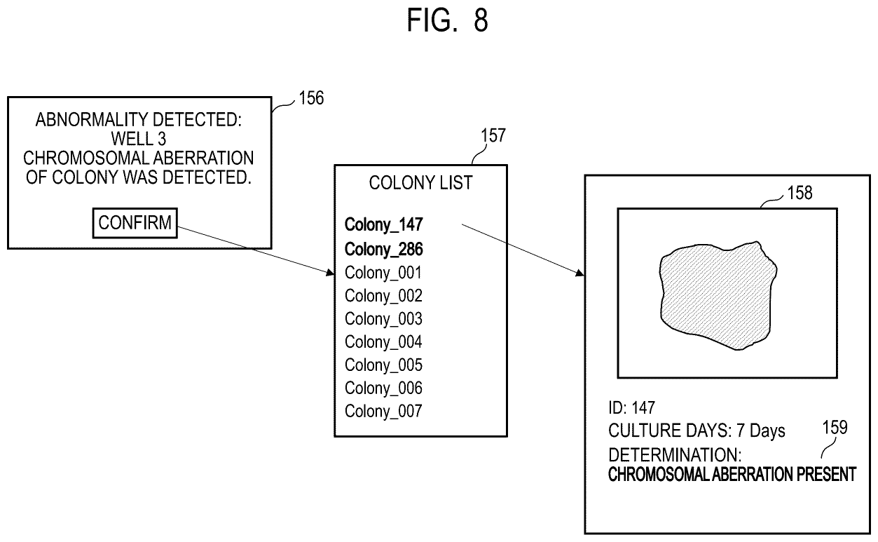

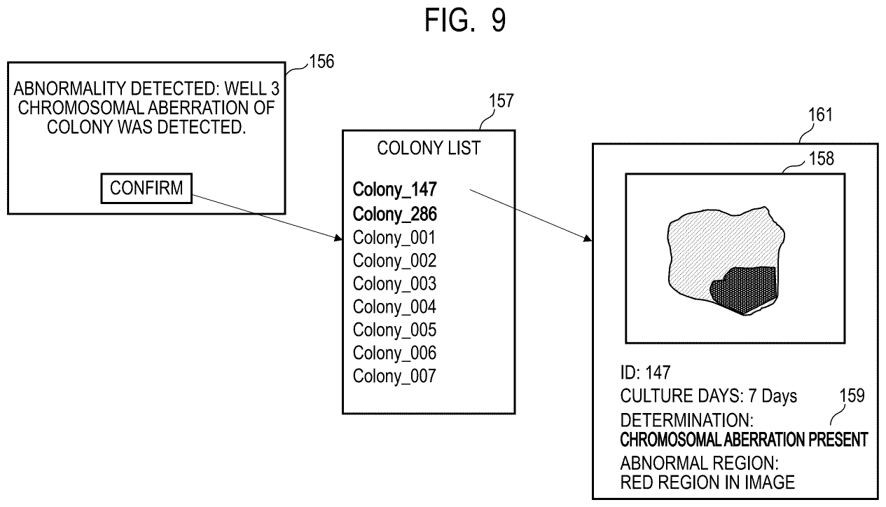

In step S45, the information processing apparatus 10 transmits the second information (presence/absence of chromosomal aberration) acquired by the second information acquiring section 153 and the image data and supplementary information acquired in step S43 to the display section 120 to notify the user. FIG. 8 and FIG. 9 are examples of screens for notifying the user on the display section 120. As illustrated in FIG. 8, the display control section 160 first displays on the display section 120 information 156 based on the second information, indicating that a chromosomal aberration of a colony has been detected, etc., on a screen such as a pop-up window. When the user presses a button such as a confirmation button, information 157 (here, a colony number) indicating the colony of pluripotent stem cells for which the analysis was performed (the second information was acquired) is displayed in a list format on the display section 120. At this time, the display control section 160 may display the information indicating the colony of pluripotent stem cells determined to have a chromosomal aberration based on the second information in a different display format from the information indicating the cell colony determined not to have a chromosomal aberration, such as by highlighting it. It can be displayed distinguishably by displaying it in a different display format. Here, displaying in a different display format means, for example, displaying in a different color. Then, when the user presses a colony number on the list, the display section 120 may display an image 158 including the colony of pluripotent stem cells, supplementary information associated with the image data (in FIG. 8, the number of days of culture), and information 159 based on the second information, such as the determination result of whether or not it has a chromosomal aberration (in FIG. 8, the information “chromosomal aberration present”). Based on the second information, the user appropriately handles the situation, such as by classifying the cell colony included in the image data including the colony of pluripotent stem cells (for example, classifying a cell colony with a chromosomal aberration and a cell colony without a chromosomal aberration among the colonies of pluripotent stem cells included in the image data) or eliminating the cell colony with a chromosomal aberration from the manufactured cells. Here, the information based on the second information is a concept that includes both the second information and information generated from the second information.

The display format and the type of information displayed are not limited to the examples described above, and other display formats or information may be displayed. FIG. 9 exemplifies a screen 161 that includes, as a variation of the display format, an image displayed by superimposing on the original image an estimated region (a region having a chromosomal aberration cell), which was estimated as a region having a chromosomal aberration cell based on the second information, among the analyzed colonies of pluripotent stem cells, as the second information in step S43. By pressing a time-series graph button (not shown) displayed on the screen 161, for example, a time-series graph showing the analysis results for the colony of pluripotent stem cells may be displayed. As another variation of the display format, a result of visualizing the basis for the determination of the second information with a heatmap or the like using a model's determination basis visualization means such as Grad-CAM technology may be superimposed on the original image and displayed.

As described above, the information processing apparatus 10 and the cell culture apparatus 40 according to the present embodiment acquire and analyze time-series data by, for example, periodically capturing an image including a cell colony being cultured on the cell culture apparatus 40, thereby making it possible to quickly detect when an abnormality occurs in the cells being cultured.

Although some embodiments have been described, these embodiments have been presented as representative examples and are not intended to limit the scope of the present disclosure. These embodiments can be implemented in various other forms as modifications thereof, and various omissions, substitutions, changes, and combinations of embodiments can be made without departing from the gist of the disclosed technology. In other words, these embodiments and their modifications are all included within the scope of the content described in the claims and the equivalents thereof.

### EXAMPLES

Hereinafter, a more specific explanation will be provided using examples. The scope of the present disclosure is not limited to the following examples. In the following examples, the research iPS cell line (Ff-I14s04) was used. For the culture protocol, the protocol “Establishment and Maintenance Culture of Human iPS Cells under Feeder-Free Conditions” published by CiRA-F was followed.

### First Example

In this example, a 6-well plate was used as the culture vessel, which was pre-coated with Laminin-511 E8 at 4.8 μg/well according to the protocol. iPS cells cultured for 7 days were detached and collected according to the protocol, and 13,000 viable cells were seeded onto the coated 6-well plate and cultured in an incubator at a temperature of 37° C. and a CO₂ concentration of 5%. The day after seeding, the medium was replaced with a maintenance culture medium (StemFit AK03N) not containing Y-27632, and thereafter the medium was replaced once every two days, and the culture was continued until the next passage.

Cells known in advance to have chromosome 12 trisomy and chromosome 12disomy were cultured, with three clones each, and images were captured in three groups, with each group being a pair of one clone of chromosome 12 trisomy and one clone of chromosome 12 disomy, to acquire data. A BZ-X810 from Keyence Corporation was used for imaging, and Tiling imaging and medium exchange were performed every 24 hours from the 5th day of culture, and image data including colonies was acquired from the 5th to the 9th day of culture.

As training data, images of individual cell colonies were extracted from the captured images by performing edge extraction on each cell colony, and 4942 images of chromosome 12 trisomy colonies and 4666 images of chromosome 12 disomy colonies were collected. As accuracy verification data, 4519 images of chromosome 12 trisomy colonies and 4392 images of chromosome 12 disomy colonies were collected, and these were used to perform learning and accuracy verification in each group.

In this example, a multitask model is used that, for a learned model having a feature vector that separates trisomy colonies and disomy colonies acquired by inputting accuracy verification colony images, outputs a chromosome 12 disomy or trisomy determination for the input colony as the second information. This model is based on ResNet18, applies Additive Angular Margin Loss to the feature vector of the intermediate layer just before the output layer, applies CrossEntropyLoss for the chromosome 12 disomy/trisomy determination in the output layer, and is a model that was learned with the weighted sum of the respective loss functions as the final loss function.

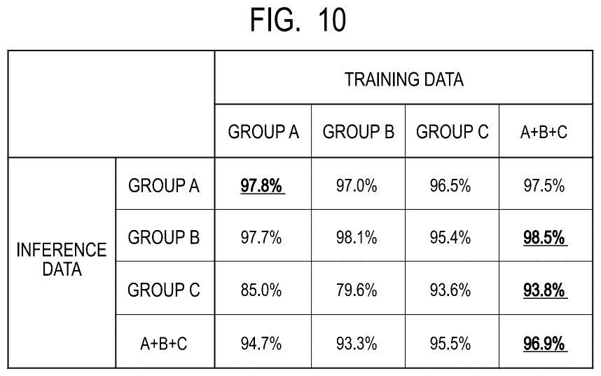

FIG. 10 is a confusion matrix describing the Accuracy of the estimation results when performing chromosome 12 disomy/trisomy determination on the accuracy verification data of each group, for the learning model of each group trained under the above conditions. The model trained with data from all groups is able to classify chromosome 12 trisomy with an accuracy of 95% or higher (as described above, groups A, B, and C each show a different clone of chromosome 12 trisomy and chromosome 12 disomy). As can be seen from FIG. 10, when inferring an untrained abnormal clone, the inference accuracy improves if the model is trained with images including colonies of cells of that clone.

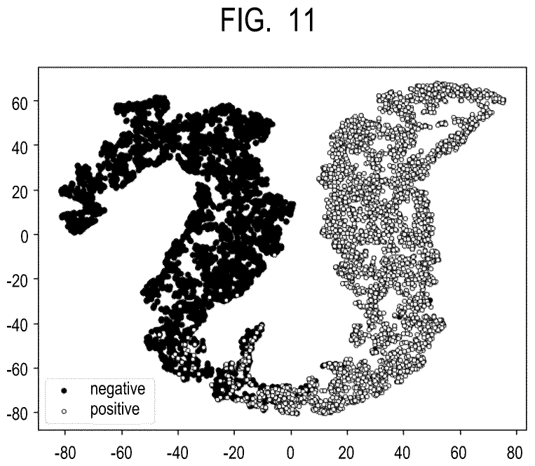

FIG. 11 is a diagram in which the feature vector of the intermediate layer to which Additive Angular Margin Loss is applied is plotted in a two-dimensional space using t-SNE. As illustrated in FIG. 11, in this model, at the stage of the feature vector, which is the first information, the morphological features obtained from the images of chromosome 12 disomy and trisomy colonies, respectively, can be recognized and separated on the feature space.

### Second Example

In this example, cells known in advance to have chromosome 12 trisomy and chromosome 12 disomy were cultured, with two clones each, and different fluorescent labels were introduced into the chromosome 12 trisomy cells and the chromosome 12 disomy cells using the PiggyBac vector system, which is one of the fluorescence introduction methods. Specifically, clones cultured for 7 days were detached and collected, respectively, suspended in D-PBS(−), and 200,000 viable cells were dispensed into a 1.5 mL tube. After centrifuging at 160 G for 5 minutes, the supernatant was removed, a plasmid solution was added thereto, and after resuspension, the mixture was transferred to a cuvette and electroporation was performed. A NEPA21 from Nepa Gene Co., Ltd. was used for the electroporation. The plasmid solution refers to a plasmid solution in which two types of plasmid vectors (a transposon plasmid and a transposase (PBase) expression helper plasmid) and Opti-MEM medium are mixed. In this example, two types of transposon plasmids were used: a plasmid designed to express puromycin as a drug resistance gene and a GFP gene as a fluorescence gene (hereinafter, GFP plasmid), and a plasmid designed to express an mCherry fluorescence gene instead of GFP (mCherry plasmid). The GFP expression plasmid was introduced into the chromosome 12 trisomy cells, and the mCherry plasmid was introduced into the chromosome 12 disomy cells. The cells into which the fluorescence gene was introduced by the above method were collected from the cuvette, seeded onto a 6-well plate pre-coated with Laminin-511 E8 that had been prepared in advance, and cultured. The day after seeding, the medium was replaced with a maintenance culture medium (StemFit AK03N) not containing Y-27632, and thereafter the medium was replaced once every two days, and the culture was continued until the next passage. From the day after the next passage, by adding puromycin to the maintenance medium, only cells into which the plasmid had been introduced were cultured. Two weeks or more after the fluorescence introduction, single-cell cloning was performed, and clones in which the fluorescence was stably and uniformly introduced were selected to obtain fluorescence-introduced clones. In this example, the fluorescence is localized in the cytoplasm.

For the fluorescence-introduced clones obtained by the method above, images were captured and data was acquired in two groups, with each group being a pair of one clone of chromosome 12 trisomy and one clone of chromosome 12 disomy. At the time of data shooting, a 6-well plate was used as the culture vessel, which was pre-coated with Laminin-511 E8 at 4.8 μg/well according to the protocol. iPS cells (fluorescence-introduced clones) cultured for 7 days were detached and collected according to the protocol, and 6,500 viable cells each were seeded onto a coated 6-well plate so that chromosome 12 trisomy and chromosome 12 disomy were mixed at a 1:1 ratio (a total of 13,000 viable cells of chromosome 12 trisomy and disomy were seeded), and cultured in an incubator at a temperature of 37° C. and a CO₂ concentration of 5%. On the first day of culture, which was the day after seeding, the medium was replaced with a maintenance culture medium (StemFit AK03N) not containing Y-27632. After seeding, Tiling imaging and medium exchange were performed every 24 hours, and image data including colonies was acquired from the 1st to the 7th day of culture. A BZ-X810 from Keyence Corporation was used for imaging, and along with phase-contrast images, fluorescence images (GFP image, TRI-TC image) were also acquired to obtain ground truth images, and 20,867 images each of the phase-contrast image and each fluorescence image were acquired.

For each fluorescence image acquired from a colony image having a disomy region and a trisomy region, to be used for machine learning training data, after binarizing the pixel values of each fluorescence image, it was processed as a ground truth label. More specifically, for each fluorescence image, a pixel value histogram was obtained, and by a binarization process that automatically set a threshold based on the peak pixel values of the non-fluorescent region and the fluorescent region of the pixel value histogram, respective mask images were generated that separated the fluorescent region from the background, and a dataset was created by combining a ground truth label image, in which a mask representing the disomy region and a mask representing the trisomy region were superimposed with different label values, and the corresponding phase-contrast image, and learning and accuracy verification were performed.

In this example, as a machine learning architecture, an Encoder-Decoder model based on U-Net was used, which extracts a feature vector representing the features of the colony morphology in each image region by applying a convolutional filter as the first information from an input image including a colony, and outputs the disomy colony region and trisomy colony region in the input image as the second information based on the first information, and Dice Loss was applied as the loss function.

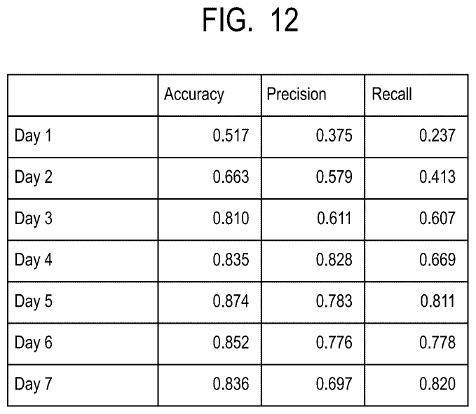

FIG. 12 is a table describing, for each number of days of culture, the Accuracy for each region of the estimation results and the Precision and Recall in the trisomy region determination when performing determination of the chromosome 12 disomy/trisomy region on the accuracy verification data, for a learning model trained under the above conditions. The Accuracy continues to improve from Day 1 to Day 4, and from Day 5 onward, region estimation is stably performed with an accuracy of 83% or higher. In this example, because the morphological feature information of each colony is extracted by the Encoder-Decoder model, it is considered that the estimation accuracy stabilized from around Day 5 (when the colony area is 50,000 μm² or more), when the morphological features of the colony become stable as the colony grows.

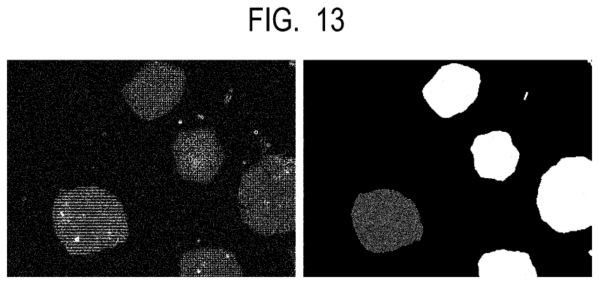

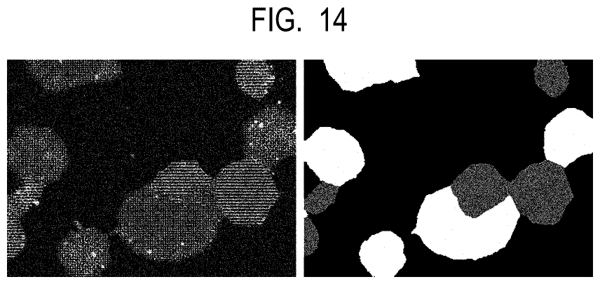

FIG. 13 and FIG. 14 are examples of determination results when performing determination of the chromosome 12 disomy/trisomy region on the aforementioned accuracy verification data. The left diagrams in FIG. 13 and FIG. 14 are diagrams in which the disomy/trisomy regions obtained from the corresponding fluorescence images are superimposed with a dot pattern/stripe pattern, respectively, on the input phase-contrast image, and the right diagrams are diagrams representing the estimated regions determined by the learned model as disomy/trisomy regions with white/gray, respectively. FIG. 13 shows a result of inference on an image that includes a group of colonies that grew independently, indicating that a correct inference was made for each colony. FIG. 14 shows a result of inference on an image that includes a colony where a part of the colonies fused and grew, indicating that even for a colony where a disomy region and a trisomy region are mixed, each region can be relatively estimated.

In the above embodiments and examples, pluripotent stem cells were used, but the above embodiments and examples can be applied to any cells that form colonies and for which the morphological features of a colony of cells having a chromosomal aberration and a colony of cells not having a chromosomal aberration are different.

The present disclosure includes an information processing apparatus, an information processing system, an information processing method, a program, and a cell manufacturing method having the following configurations or methods.

### (Configuration 1)

An information processing apparatus, comprising: a first information acquiring section that acquires first information, which is information regarding morphological features of a colony of pluripotent stem cells included in first image data, obtained by inputting the first image data including a colony of pluripotent stem cells to a learned model obtained by learning with a dataset including image data of a first image group including a colony of pluripotent stem cells having a chromosomal aberration, and image data of a second image group including a colony of pluripotent stem cells not having a chromosomal aberration; and a second information acquiring section that acquires, based on the first information, second information, which is information regarding a chromosomal aberration of the colony of pluripotent stem cells included in the first image data.

### (Configuration 2)

The information processing apparatus according to configuration 1, wherein the morphological features of the colony of pluripotent stem cells are morphological features common to the colonies of pluripotent stem cells included in a plurality of images of a same image group in the dataset.

### (Configuration 3)

The information processing apparatus according to configuration 1 or 2, wherein the first information acquiring section acquires the first information by a statistical value based on an analysis regarding the colony of pluripotent stem cells included in the first image data being input to the learned model together with the first image data.

### (Configuration 4)

The information processing apparatus according to any one of configurations 1 to 3, wherein the first information acquiring section acquires the first information by a scalar value regarding a culture state of the colony of pluripotent stem cells included in the first image data being input to the learned model together with the first image data.

### (Configuration 5)

The information processing apparatus according to configuration 1 or 2, wherein the second information acquiring section acquires the second information based on the first information and a statistical value based on an analysis regarding the colony of pluripotent stem cells included in the first image data.

### (Configuration 6)

The information processing apparatus according to configuration 1 or 2, wherein the second information acquiring section acquires the second information based on the first information and a scalar value regarding a culture state of the cell colony included in the first image data.

### (Configuration 7)

The information processing apparatus according to any of configurations 1 to 6, wherein the first information includes at least one of a feature vector of a cell colony included in the first image data, a score at each pixel of the first image data, and a probability value at each pixel of the first image data.

### (Configuration 8)

The information processing apparatus according to any one of configurations 1 to 7, further comprising an image data input section that inputs the first image data to the learned model; and a first information output section from which the first information is output from the learned model.

### (Configuration 9)

The information processing apparatus according to any one of configurations 1 to 8, further comprising an image data acquiring section that acquires the first image data.

### (Configuration 10)

The information processing apparatus according to any one of configurations 1 to 9, wherein the acquisition of the first information by the first information acquiring section and the acquisition of the second information by the second information acquiring section are performed sequentially by one learned model.

### (Configuration 11)

The information processing apparatus according to any one of configurations 1 to 10, wherein the first image group and the second image group are both image groups imaged time-serially,
the first image data including the colony of pluripotent stem cells, which is input to the learned model, is time-series image data of an image including the colony of pluripotent stem cells, and
the morphological features of the cell colony included in the image data including the colony of pluripotent stem cells are morphological features of the colony of pluripotent stem cells included in the time-series image data.

### (Configuration 12)

The information processing apparatus according to any one of configurations 1 to 11, wherein the second information includes information regarding a determination of whether or not the colony of pluripotent stem cells included in the first image data has a chromosomal aberration.

### (Configuration 13)

The information processing apparatus according to any one of configurations 1 to 12, wherein the second information includes information regarding a region indicating a chromosomal aberration of the colony of pluripotent stem cells included in the first image data.

### (Configuration 14)

The information processing apparatus according to any one of configurations 1 to 11, wherein the first information acquiring section acquires first information regarding the colony of pluripotent stem cells for each of different positions by inputting an image group of the colonies of pluripotent stem cells at different positions included in a same medium culture to the learned model, and the second information acquiring section acquires as the second information a statistical value of information regarding a determination of whether or not a chromosomal aberration is present for the colony of pluripotent stem cells at each of the different positions.

### (Configuration 15)

The information processing apparatus according to any one of configurations 1 to 14, having a display control section, wherein the display control section performs control to display information based on the second information on a display section.

### (Configuration 16)

The information processing apparatus according to configuration 15, wherein the display control section performs control to display information based on the first information on the display section together with the information based on the second information.

### (Configuration 17)

An information processing apparatus according to any one of configurations 1 to 16, having a display control section, wherein the display control section displays, based on the second information acquired by the second information acquiring section, information indicating a colony of pluripotent stem cells determined to have a chromosomal aberration cell and information indicating a colony of pluripotent stem cells determined not to have a chromosomal aberration cell in different display formats.

### (Configuration 18)

The information processing apparatus according to configuration 17, wherein the display in the different display format is a display in a different color.

### (Configuration 19)

The information processing apparatus according to any one of configurations 1 to 18, wherein the display control section, based on the second information acquired by the second information acquiring section, displays on a display section a superimposed image of an image indicating a region estimated to have a chromosomal aberration among the colonies of pluripotent stem cells, and an image including the colony of pluripotent stem cells.

### (Configuration 20)

An information processing system, comprising:
an image data acquiring section that acquires first image data including a colony of pluripotent stem cells;
a first information acquiring section that acquires first information, which is information regarding morphological features of the colony of pluripotent stem cells included in the first image data, by inputting the first image data acquired by the image data acquiring section to a learned model obtained by learning with a dataset including image data of a first image group including a colony of pluripotent stem cells having a chromosomal aberration, and image data of a second image group including a colony of pluripotent stem cells not having a chromosomal aberration; and
a second information acquiring section that acquires, based on the first information, second information, which is information regarding a chromosomal aberration of the colony of pluripotent stem cells included in the first image data.

### (Configuration 21)

The information processing system according to configuration 20, wherein the morphological features of the colony of pluripotent stem cells are morphological features common to the colonies of pluripotent stem cells included in a plurality of images of a same image group in the dataset.

### (Configuration 22)

The information processing system according to configuration 20 or 21, wherein the first information acquiring section acquires the first information by a statistical value based on an analysis regarding the colony of pluripotent stem cells included in the first image data being input to the learned model together with the first image data.

### (Configuration 23)

The information processing system according to any one of configurations 20 to 22, wherein the first information acquiring section acquires the first information by a scalar value regarding a culture state of the colony of pluripotent stem cells included in the first image data being input to the learned model together with the first image data.

### (Configuration 24)

The information processing system according to configuration 20 or 21, wherein the second information acquiring section acquires the second information based on the first information and a statistical value based on an analysis regarding the colony of pluripotent stem cells included in the first image data.

### (Configuration 25)

The information processing system according to configuration 20 or 21, wherein the second information acquiring section acquires the second information based on the first information and a scalar value regarding a culture state of the colony of pluripotent stem cells included in the first image data.

### (Configuration 26)

The information processing system according to configuration 20, wherein the first information includes at least one of a feature vector of a cell colony included in the first image data, a score at each pixel of the first image data, and a probability value at each pixel of the first image data.

### (Configuration 27)

The information processing system according to any one of configurations 20 to 26, further comprising an image data input section that inputs the first image data to the learned model; and a first information output section from which the first information is output from the learned model.

### (Configuration 28)

The information processing system according to any one of configurations 20 to 27, further comprising an image data acquiring section that acquires the first image data.

### (Configuration 29)

The information processing system according to any one of configurations 20 to 28, wherein the acquisition of the first information by the first information acquiring section and the acquisition of the second information by the second information acquiring section are performed sequentially by one learned model.

### (Configuration 30)

The information processing system according to any one of configurations 20 to 29, wherein the first image group and the second image group are both image groups imaged time-serially,
the first image data including the colony of pluripotent stem cells, which is input to the learned model, is time-series image data of an image including the colony of pluripotent stem cells, and
the morphological features of the cell colony included in the image data including the colony of pluripotent stem cells are morphological features of the colony of pluripotent stem cells included in the time-series image data.

### (Configuration 31)

The information processing system according to any one of configurations 20 to 30, wherein the second information includes information regarding a determination of whether or not the colony of pluripotent stem cells included in the first image data has a chromosomal aberration.

### (Configuration 32)

The information processing system according to any one of configurations 20 to 30, wherein the second information includes information regarding a region indicating a chromosomal aberration of the colony of pluripotent stem cells included in the first image data.

### (Configuration 33)

The information processing system according to any one of configurations 20 to 31, wherein the first information acquiring section acquires first information regarding the colony of pluripotent stem cells for each of different positions by inputting an image group of the colonies of pluripotent stem cells at different positions included in a same medium culture to the learned model, and the second information acquiring section acquires as the second information a statistical value of information regarding a determination of whether or not a chromosomal aberration is present for the colony of pluripotent stem cells at each of the different positions.

### (Configuration 34)

The information processing system according to any one of configurations 20 to 33, having a display control section, wherein the display control section performs control to display information based on the second information on a display section.

### (Configuration 35)

The information processing system according to configuration 34, wherein the display control section performs control to display information based on the first information on the display section together with the information based on the second information.

### (Configuration 36)

The information processing system according to any one of configurations 20 to 35, having a display control section, wherein the display control section displays, based on the second information acquired by the second information acquiring section, information indicating a colony of pluripotent stem cells determined to have a chromosomal aberration cell and information indicating a colony of pluripotent stem cells determined not to have a chromosomal aberration cell in different display formats.

### (Configuration 37)

The information processing system according to configuration 36, wherein the display in the different display format is a display in a different color.

### (Configuration 38)

The information processing system according to any one of configurations 20 to 35, wherein the information processing system includes a display control section, and the display control section, based on the second information acquired by the second information acquiring section, displays on a display section a superimposed image of an image indicating a region estimated to have a chromosomal aberration among the colonies of pluripotent stem cells, and an image including the colony of pluripotent stem cells.

### (Method 1)

An information processing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration; and
a second information acquiring step of acquiring, based on the first information, second information, which is information regarding a chromosomal aberration of the colony of pluripotent stem cells included in the first image data.

### (Program 1)

A program that, when read by a computer, causes the computer to execute each step of the information processing method according to method 1.

### (Manufacturing Method 1)

A cell manufacturing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
a classifying step of classifying the colony of pluripotent stem cells included in the first image data, based on the second information.

### (Manufacturing Method 2)

A cell manufacturing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model obtained by learning with a dataset including image data of a first image group including a colony of pluripotent stem cells having a chromosomal aberration, and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
an eliminating step of eliminating a cell colony having a chromosomal aberration from the colony of pluripotent stem cells included in the first image data, based on the second information.

Regarding the acquisition of the second information in (Configuration 1), it can also be expressed by the following (Configuration 39).

### (Configuration 39)

An information processing apparatus having a second information acquiring section that acquires second information, which is information regarding a chromosomal aberration of a colony of pluripotent stem cells included in first image data, based on first information, which is information regarding morphological features of the colony of pluripotent stem cells included in the first image data, obtained by inputting the first image data including a colony of pluripotent stem cells to a learned model obtained by learning with a dataset including image data of a first image group including a colony of pluripotent stem cells having a chromosomal aberration, and image data of a second image group including a colony of pluripotent stem cells not having a chromosomal aberration.

According to the technology of the present disclosure, it is possible to non-invasively and efficiently detect chromosomal aberrations in cell colonies.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-143443, filed Aug. 23, 2024, and Japanese Patent Application No. 2025-044614, filed Mar. 19, 2025, which are hereby incorporated by reference herein in their entirety.

What is claimed is:

1. An information processing system, comprising:
an image data acquiring section that acquires first image data including a colony of pluripotent stem cells;
a first information acquiring section that acquires first information, which is information regarding morphological features of the colony of pluripotent stem cells included in the first image data, by inputting the first image data acquired by the image data acquiring section to a learned model, which is obtained by learning with a dataset including image data of a first image group including a colony of pluripotent stem cells having a chromosomal aberration and image data of a second image group including a colony of pluripotent stem cells not having a chromosomal aberration; and
a second information acquiring section that acquires, based on the first information, second information, which is information regarding chromosomal aberration of the colony of pluripotent stem cells included in the first image data.

2. The information processing system according to claim 1, wherein the morphological features of the colony of pluripotent stem cells are morphological features common to colonies of pluripotent stem cells included in a plurality of images of a same image group in the dataset.

3. The information processing system according to claim 1, wherein the first information acquiring section acquires the first information by inputting a statistical value based on an analysis regarding the colony of pluripotent stem cells included in the first image data to the learned model together with the first image data.

4. The information processing system according to claim 1, wherein the first information acquiring section acquires the first information by inputting a scalar value regarding a culture state of the colony of pluripotent stem cells included in the first image data to the learned model together with the first image data.

5. The information processing system according to claim 1, wherein the second information acquiring section acquires the second information based on the first information and a statistical value based on an analysis regarding the colony of pluripotent stem cells included in the first image data.

6. The information processing system according to claim 1, wherein the second information acquiring section acquires the second information based on the first information and a scalar value regarding a culture state of the colony of pluripotent stem cells included in the first image data.

7. The information processing system according to claim 1, wherein the first information includes at least one of a feature vector of the colony included in the first image data, a score at each pixel of the first image data, and a probability value at each pixel of the first image data.

8. The information processing system according to claim 1, further comprising an image data input section that inputs the first image data to the learned model and a first information output section from which the first information is output from the learned model.

9. The information processing system according to claim 1, further comprising an image data acquiring section that acquires the first image data.

10. The information processing system according to claim 1, wherein the acquisition of the first information by the first information acquiring section and the acquisition of the second information by the second information acquiring section are performed sequentially by a single learned model.

11. The information processing system according to claim 1, wherein
each of the first image group and the second image group includes images captured time-serially,
the first image data to be input to the learned model is time-series image data of an image including the colony of pluripotent stem cells, and
the morphological features of the colony of pluripotent stem cells are included in the time-series image data.

12. The information processing system according to claim 1, wherein the second information includes information regarding determination of whether or not the colony of pluripotent stem cells included in the first image data has a chromosomal aberration.

13. The information processing system according to claim 1, wherein the second information includes information regarding a region indicating a chromosomal aberration of the colony of pluripotent stem cells included in the first image data.

14. The information processing system according to claim 1, wherein the first information acquiring section acquires the first information for each of colonies of pluripotent stem cells at different positions by inputting image data for each of the colonies at different positions included in a same medium culture to the learned model, and the second information acquiring section acquires as the second information a statistical value of information regarding determination of whether or not a chromosomal aberration is present for each of the colonies at different positions.

15. The information processing system according to claim 1, wherein the information processing system includes a display control section, and the display control section performs control to display information on a display section based on the first information together with information based on the second information.

16. The information processing system according to claim 15, wherein the display control section, based on the second information acquired by the second information acquiring section, displays on a display section a superimposed image composed of an image indicating a region estimated to have chromosomal aberration in the colony of pluripotent stem cells and an image including the colony of pluripotent stem cells.

17. An information processing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model, which is obtained by learning with a dataset including image data of a first image group including a colony of pluripotent stem cells having a chromosomal aberration and image data of a second image group including a colony of pluripotent stem cells not having a chromosomal aberration; and
a second information acquiring step of acquiring, based on the first information, second information, which is information regarding a chromosomal aberration of the colony of pluripotent stem cells included in the first image data.

18. A non-transitory computer-readable storage medium storing a program that, when read by a computer, causes the computer to execute each step of the information processing method according to claim 17.

19. A cell manufacturing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model, which is obtained by learning with a dataset including image data of a first image group including a colony of pluripotent stem cells having a chromosomal aberration and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
a classifying step of classifying the colony of pluripotent stem cells included in the first image data, based on the second information.

20. A cell manufacturing method, comprising:
an image data acquiring step of acquiring first image data including a colony of pluripotent stem cells;
a first information acquiring step of acquiring first information, which is information regarding morphological features of the colony of pluripotent stem cells included in the first image data, by inputting the first image data acquired in the image data acquiring step to a learned model, which is obtained by learning with a dataset including image data of a first image group including a colony of pluripotent stem cells having a chromosomal aberration, and image data of a second image group including a colony of pluripotent stem cells not having a chromosomal aberration;
a second information acquiring step of acquiring, based on the first information, second information, which is information regarding a chromosomal aberration of the colony of pluripotent stem cells included in the first image data; and
an eliminating step of eliminating a cell colony having a chromosomal aberration from the colony of pluripotent stem cells included in the first image data, based on the second information.
